(19) United States
(12) Patent Application Publication
(10) Pub. No.: US 2008/0242994 A1
(43) Pub. Date: Oct. 2, 2008

(54) METHODS AND APPARATUS FOR ULTRASOUND IMAGING

(75) Inventor: Tadashi Tamura, North Haven, CT (US)

Correspondence Address: BACHMAN & LAPOINTE, P.C., 900 CHAPEL STREET, SUITE 1201, NEW HAVEN, CT, 06510, US

(73) Assignee: ALOKA CO., LTD., Tokyo (JP)

(21) Appl. No.: 11/926,251

(22) Filed: Oct. 29, 2007

Related U.S. Application Data
(60) Provisional application No. 60/921,089, filed on Mar. 29, 2007.

(51) Int. Cl. A61B 8/00

(52) U.S. Cl. 600/453

(57) ABSTRACT

The maximum frequency in a Doppler spectrum is obtained and used as an aliasing detector. When aliasing occurs, frequencies greater than a frequency limit change from one frequency region to another. When aliasing is detected, a zero frequency baseline is shifted to prevent future aliasing.

### CROSS REFERENCE TO RELATED APPLICATIONS

This application claims the benefit of U.S. Provisional Application No. 60/921,089, filed on Mar. 29, 2007, the disclosure which is incorporated herein by reference in its entirety.

### BACKGROUND OF THE INVENTION

The invention relates generally to the field of ultrasound imaging. More specifically, embodiments of the invention relate to methods and systems for spectral images.

Ultrasound is used to image various organs, heart, liver, fetus, and blood vessels. For diagnosis of cardiovascular diseases, spectral Doppler is usually used to measure blood flow velocity. The pulsed spectral Doppler technique is usually used as it has spatial sampling capability which permits the sampling of velocity in a blood vessel compared with the continuous wave (CW) technique which does not have spatial discrimination capability and samples all signals along the ultrasound beam.

In a Doppler technique, the ultrasound is transmitted at a pulse repetition frequency (PRF) and the blood flow velocity is detected as the shift in frequency (Doppler shift frequency) in the received ultrasound signal. The received ultrasound is mixed with in-phase (0 degrees) and quadrature (90 degrees) reference signals of the same frequency as the transmit ultrasound frequency. After low-pass filtering high frequency components (i.e. second harmonics), only the baseband signals are obtained. Wall-filtering (e.g. highpass filtering) is applied to the baseband signals to remove strong clutter noise from tissue and slowly moving tissues such as blood vessel walls, resulting in complex I-Q Doppler signals.

Generally, the I-Q Doppler signals are input to a spectrum analyzer such as a Fast Fourier Transform (FFT) to obtain the Doppler spectrum which represents the blood velocities. The Doppler shift frequency and the blood velocity have the following relationship

$$Δ f = \frac{2 {f}_{t} v cos θ}{c} , \qquad ( 1 )$$

where Δf is the Doppler shift frequency, fₜ is the transmitted frequency, v is the blood velocity, θ is the angle between the ultrasound beam direction and the velocity vector and c is the speed of sound.

128-point, 256-point or 512-point fast Fourier Transforms (FFTs) are often used. Because the Doppler signals are obtained by the pulsed ultrasound (and also sampling) technique, sampling theory dictates a maximum frequency limit. The maximum frequency is generally half of the pulse repetition frequency (PRF) or f_{PRF}. Since an FFT is performed on the complex I-Q Doppler signals, blood flow velocity in a negative direction appears in the negative frequency domain. Therefore, the Doppler spectrum FFT output has negative frequencies that correspond to negative velocities. Thus, the Doppler spectrum usually has a range of

$$- \frac{{f}_{PRF}}{2} to \frac{{f}_{PRF}}{2}$$

in frequency. However, the negative frequency range may be allocated to represent the positive frequency of more than

$$\frac{{f}_{PRF}}{2}$$

and up to f_{PRF}. In the opposite case, the positive frequency range may be allocated to represent the negative frequency of less than

$$- \frac{{f}_{PRF}}{2}$$

and up to −f_{PRF}. In the Doppler spectrum mode, this is performed by a baseline shift. A baseline shift moves the position of a zero frequency baseline in either a positive or negative frequency direction. Thus, the Doppler spectrum may have a range from −f_{PRF} to 0, or from 0 to f_{PRF} at extreme cases due to baseline shifting. The all frequency range is always f_{PRF}.

Often in cardiovascular applications, blood velocities may exceed these maximum velocities, resulting in aliasing. When aliasing occurs, the frequency spectrum may wrap around at the positive maximum frequency, with frequencies exceeding the maximum limit appearing in the negative frequencies, or wrap around at the negative maximum frequency, with frequencies exceeding the negative maximum limit appearing in the positive frequencies. Aliasing makes blood velocity determination difficult.

Conversely, the f_{PRF} may be too large to measure blood velocity accurately. The maximum blood flow velocity (maximum frequency) may be only about one tenth of the maximum frequency limit which would make the displayed spectrum too small to accurately measure.

In most ultrasound applications, a user manually adjusts the PRF, which corresponds to blood velocity, and/or a baseline which is the zero frequency position which corresponds to zero velocity in the frequency spectrum scale. However, in adjusting these settings, the user consumes time that would be better spent in diagnosis.

There exists a need to overcome these problems.

### SUMMARY OF THE INVENTION

The inventor has discovered that it would be desirable to have a system and method where the maximum frequency in a Doppler spectrum is obtained and used as an aliasing detector. When aliasing occurs, the maximum frequencies wrap from a positive frequency to a negative frequency, or from a negative frequency to a positive frequency. When aliasing is detected, the baseline is shifted to accommodate the magnitudes of the wrapped frequencies in the correct frequency polarity.

One aspect of the invention provides methods for detecting and correcting aliasing in a Doppler frequency spectrum. Methods according to this aspect of the invention comprise receiving a Doppler frequency spectrum signal over time, calculating maximum frequencies fₘₐₓ and minimum frequencies fₘᵢₙ from the Doppler frequency spectra, tracking the maximum fₘₐₓ and minimum fₘᵢₙ frequencies over time, detecting whether aliasing is occurring from the maximum frequencies fₘₐₓ if frequencies in a positive frequency region change (wrap) to a negative frequency region, or detecting whether aliasing is occurring from the minimum frequencies fₘᵢₙ if negative frequencies in the negative frequency region change (wrap) to the positive region, and if aliasing is detected, shifting a zero frequency baseline separating the negative and positive frequency regions of the Doppler spectrum in either a positive or negative direction according to a maximum frequency deviation fₐ.

Another aspect of the invention provides methods for determining a pulse repetition frequency for an ultrasound system. Methods according to this aspect of the invention comprise receiving a Doppler frequency spectrum signal over time, calculating maximum frequencies fₘₐₓ from the Doppler frequency spectra, calculating minimum frequencies fₘᵢₙ from the Doppler frequency spectra, tracking the maximum fₘₐₓ and minimum fₘᵢₙ frequencies over time, capturing a highest value high fₘₐₓ of the maximum fₘₐₓ frequencies and a lowest value low fₘᵢₙ of the minimum fₘᵢₙ frequencies tracked, comparing the highest value high fₘₐₓ and the lowest value low fₘᵢₙ to determine whether the maximum fₘₐₓ frequencies and minimum fₘᵢₙ frequencies are bipolar, or negative or positive unipolar, if bipolar: determining a frequency span based on a difference between the highest maximum frequency high fₘₐₓ and lowest minimum frequency low fₘᵢₙ, comparing the frequency span to a current PRF setting value, if the frequency span is greater than the current PRF setting value, increase the PRF setting value, if the frequency span is less than a predetermined fraction of the current PRF setting value, decrease the PRF setting value, and if the frequency span is less than the current PRF setting value but greater than the predetermined fraction of the current PRF, use the current PRF setting value, if positive unipolar: comparing the highest maximum frequency high fₘₐₓ with a current positive maximum frequency limit b₁f_{PRF}, wherein if the highest maximum frequency high fₘₐₓ is greater than the current positive maximum frequency limit b₁f_{PRF}, the current PRF setting value is increased to a setting corresponding to the highest maximum frequency high fₘₐₓ, if the highest maximum frequency high fₘₐₓ is less than a current positive maximum frequency limit b₁f_{PRF}, comparing the highest maximum frequency high fₘₐₓ with a low level threshold b₂b₁f_{PRF}, wherein if the highest maximum frequency high fₘₐₓ is less than the low level threshold b₂b₁f_{PRF}, the PRF is decreased until equal to the highest maximum frequency high fₘₐₓ, and if negative unipolar: comparing the absolute value of the lowest minimum frequency low fₘᵢₙ with the absolute value of a current negative maximum frequency limit −(1−b₁)f_{PRF}, wherein if the absolute value of the lowest minimum frequency low fₘᵢₙ is greater than the absolute value of the current negative maximum frequency limit −(1−b₁)f_{PRF}, the current PRF setting value is increased to a setting corresponding to the absolute value of the lowest minimum frequency low fₘᵢₙ, if the absolute value of the lowest minimum frequency low fₘᵢₙ is less than the absolute value of the current negative maximum frequency limit −(1−b₁)f_{PRF}, comparing the absolute value of the lowest minimum frequency low fₘᵢₙ with the absolute value of a low level threshold −b₂(1−b₁)f_{PRF}, wherein if the absolute value of the lowest minimum frequency low fₘᵢₙ is less than the absolute value of the low level threshold −b₂(1-b₁)f_{PRF}, the PRF is decreased to equal the absolute value of the lowest minimum frequency low fₘᵢₙ.

Another aspect of the invention provides methods for determining a pulse repetition frequency for an ultrasound system. Methods according to this aspect of the invention comprise setting an initial pulse repetition frequency, receiving a Doppler frequency spectrum signal over time, calculating maximum frequencies fₘₐₓ from the Doppler frequency spectra, calculating minimum frequencies fₘᵢₙ from the Doppler frequency spectra, tracking the maximum fₘₐₓ and minimum f_{min,} frequencies over time, capturing a highest value high fₘₐₓ of the maximum frequencies fₘₐₓ and a lowest value low fₘᵢₙ of the minimum frequencies fₘᵢₙ tracked, comparing the absolute value of the highest maximum value high fₘₐₓ with the absolute value of the lowest minimum frequency low fₘᵢₙ to determine whether the positive or negative frequency region takes precedence, if the highest maximum value high fₘₐₓ is greater, the positive frequency region takes precedence and a positive low level threshold b₂b₁f_{PRF} is calculated, and comparing the highest maximum frequency high fₘₐₓ with the positive maximum frequency limit b₁f_{PRF} and the positive low level threshold b₂b₁f_{PRF} wherein if the highest maximum frequency high fₘₐₓ is less than the positive low level threshold b₂b₁f_{PRF}, the PRF is decreased until the positive maximum frequency limit b₁f_{PRF} equals the highest maximum frequency high fₘₐₓ, or aliasing starts to occur at the negative maximum frequency limit −(1−b₁)f_{PRF} whichever comes first, and wherein if the highest maximum frequency high fₘₐₓ is greater than the positive maximum frequency limit b₁f_{PRF}, the PRF is increased to equal the highest maximum frequency high fₘₐₓ, if the absolute value of the lowest minimum frequency low fₘᵢₙ is greater, the negative frequency region takes precedence and a low level threshold −b₂(1−b₁)f_{PRF} is calculated, comparing the absolute value of the lowest minimum frequency low fₘᵢₙ with the absolute value of the negative maximum frequency limit −(1−b₁)f_{PRF} and the absolute value of the low level threshold −b₂(1−b₁)f_{PRF} wherein if the absolute value of the lowest minimum frequency low fₘᵢₙ is less than the absolute value of the low level threshold −b₂(1−b₁)f_{PRF}, the PRF is decreased until the absolute value of the negative maximum frequency limit −(1−b₁)f_{PRF} is the absolute value the lowest minimum frequency low fₘᵢₙ or aliasing starts to occur at the positive frequency limit whichever comes first, and wherein if the absolute value of the lowest minimum frequency low fₘᵢₙ is greater than the absolute value of the negative maximum frequency limit −(1−b₁)f_{PRF}, the PRF is increased to equal the lowest minimum frequency low fₘᵢₙ.

Another aspect of the invention provides systems for detecting and correcting aliasing in a Doppler frequency spectrum. Systems according to this aspect of the invention comprise means for receiving a Doppler frequency spectrum signal over time, means for calculating maximum frequencies fₘₐₓ and minimum frequencies fₘᵢₙ from the Doppler frequency spectra, means for tracking the maximum fₘₐₓ and minimum fₘᵢₙ frequencies over time, means for detecting whether aliasing is occurring from the maximum frequencies fₘₐₓ if frequencies in a positive frequency region change (wrap) to a negative frequency region, means for detecting whether aliasing is occurring from the minimum frequencies fₘᵢₙ if negative frequencies in the negative frequency region change (wrap) to the positive region, and if aliasing is detected, means for shifting a zero frequency baseline separating the negative and positive frequency regions of the Doppler spectrum in either a positive or negative direction according to a maximum frequency deviation fₐ.

Another aspect of the invention provides systems for determining a pulse repetition frequency for an ultrasound system. Systems according to this aspect of the invention comprise means for setting an initial pulse repetition frequency, means for receiving a Doppler frequency spectrum signal over time, means for calculating maximum frequencies fₘₐₓ from the Doppler frequency spectra, means for calculating minimum frequencies fₘᵢₙ from the Doppler frequency spectra, means for tracking the maximum fₘₐₓ and minimum fₘᵢₙ frequencies over time, means for capturing a highest value high fₘₐₓ of the maximum frequencies fₘₐₓ and a lowest value low fₘᵢₙ of the minimum frequencies fₘᵢₙ tracked, means for comparing the absolute value of the highest maximum value high fₘₐₓ with the absolute value of the lowest minimum frequency low fₘᵢₙ to determine whether the positive or negative frequency region takes precedence, if the highest maximum value high fₘₐₓ is greater, the positive frequency region takes precedence and a positive low level threshold b₂b₁f_{PRF} is calculated, and means for comparing the highest maximum frequency high fₘₐₓ with the positive maximum frequency limit b₁f_{PRF} and the positive low level threshold b₂b₁f_{PRF} wherein if the highest maximum frequency high fₘₐₓ is less than the positive low level threshold b₂b₁f_{PRF}, the PRF is decreased until the positive maximum frequency limit b₁f_{PRF} equals the highest maximum frequency high fₘₐₓ, or aliasing starts to occur at the negative maximum frequency limit −(1−b₁)f_{PRF} whichever comes first, and wherein if the highest maximum frequency high fₘₐₓ is greater than the positive maximum frequency limit b₁f_{PRF}, the PRF is increased to equal the highest maximum frequency high fₘₐₓ, if the absolute value of the lowest minimum frequency low fₘᵢₙ is greater, the negative frequency region takes precedence and a low level threshold −b₂(1−b₁)f_{PRF} is calculated, means for comparing the absolute value of the lowest minimum frequency low fₘᵢₙ with the absolute value of the negative maximum frequency limit −(1−b₁)f_{PRF} and the absolute value of the low level threshold −b₂(1−b₁)f_{PRF} wherein if the absolute value of the lowest minimum frequency low fₘᵢₙ is less than the absolute value of the low level threshold −b₂(1−b₁)f_{PRF}, the PRF is decreased until the absolute value of the negative maximum frequency limit −(1−b₁)f_{PRF} is the absolute value the lowest minimum frequency low fₘᵢₙ or aliasing starts to occur at the positive frequency limit whichever comes first, and wherein if the absolute value of the lowest minimum frequency low fₘᵢₙ is greater than the absolute value of the negative maximum frequency limit −(1−b₁)f_{PRF}, the PRF is increased to equal the lowest minimum frequency low fₘᵢₙ.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is an exemplary plot showing a maximum Doppler frequency exhibiting aliasing.

FIG. 2A is an exemplary plot showing the maximum Doppler frequency of a frequency spectrum as a percentile.

FIG. 2B is an exemplary plot showing the minimum Doppler frequency of a frequency spectrum as a percentile.

FIG. 3 is an exemplary maximum Doppler frequency plot after a corrective baseline shift.

FIG. 4 is an exemplary plot showing minimum, mean and maximum frequencies of a frequency spectrum.

FIG. 5A is an exemplary plot showing bipolar maximum and minimum frequencies of Doppler spectra.

FIG. 5B is an exemplary plot showing unipolar positive maximum and minimum frequencies of Doppler spectra.

FIG. 5C is an exemplary plot showing unipolar negative maximum and minimum frequencies of Doppler spectra.

FIG. 6 is an exemplary flow chart to describe automatic baseline shifting method.

FIG. 7 is an exemplary flow chart to describe automatic PRF setting and baseline shifting method.

FIG. 8 is an exemplary flow chart to describe automatic PRF setting with fixed baseline method.

FIG. 9 is an exemplary ultrasound system with automatic baseline shifting and PRF setting.

FIG. 10 is an exemplary Doppler spectrum over time, showing the maximum and minimum frequencies.

### DETAILED DESCRIPTION

Embodiments of the invention will be described with reference to the accompanying drawing figures wherein like numbers represent like elements throughout. Before embodiments of the invention are explained in detail, it is to be understood that the invention is not limited in its application to the details of the examples set forth in the following description or illustrated in the figures. The invention is capable of other embodiments and of being practiced or carried out in a variety of applications and in various ways. Also, it is to be understood that the phraseology and terminology used herein is for the purpose of description and should not be regarded as limiting. The use of “including,” “comprising,” or “having,” and variations thereof herein is meant to encompass the items listed thereafter and equivalents thereof as well as additional items. The terms “mounted,” “connected,” and “coupled,” are used broadly and encompass both direct and indirect mounting, connecting, and coupling. Further, “connected,” and “coupled” are not restricted to physical or mechanical connections or couplings.

It should be noted that the invention is not limited to any particular software language described or that is implied in the figures. One of ordinary skill in the art will understand that a variety of alternative software languages may be used for implementation of the invention. It should also be understood that some of the components and items are illustrated and described as if they were hardware elements, as is common practice within the art. However, one of ordinary skill in the art, and based on a reading of this detailed description, would understand that, in at least one embodiment, components in the method and system may be implemented in software or hardware.

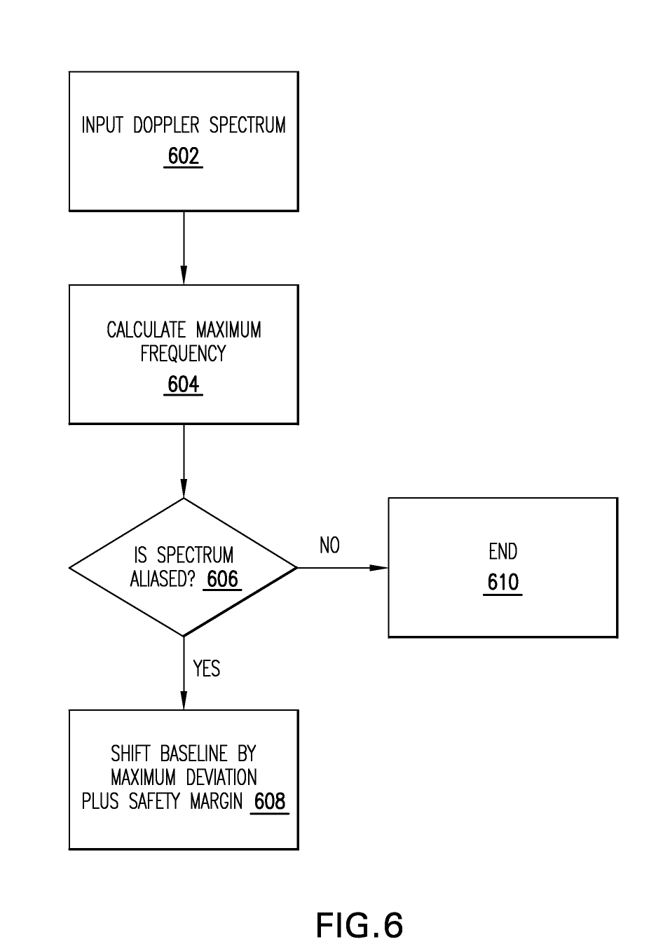

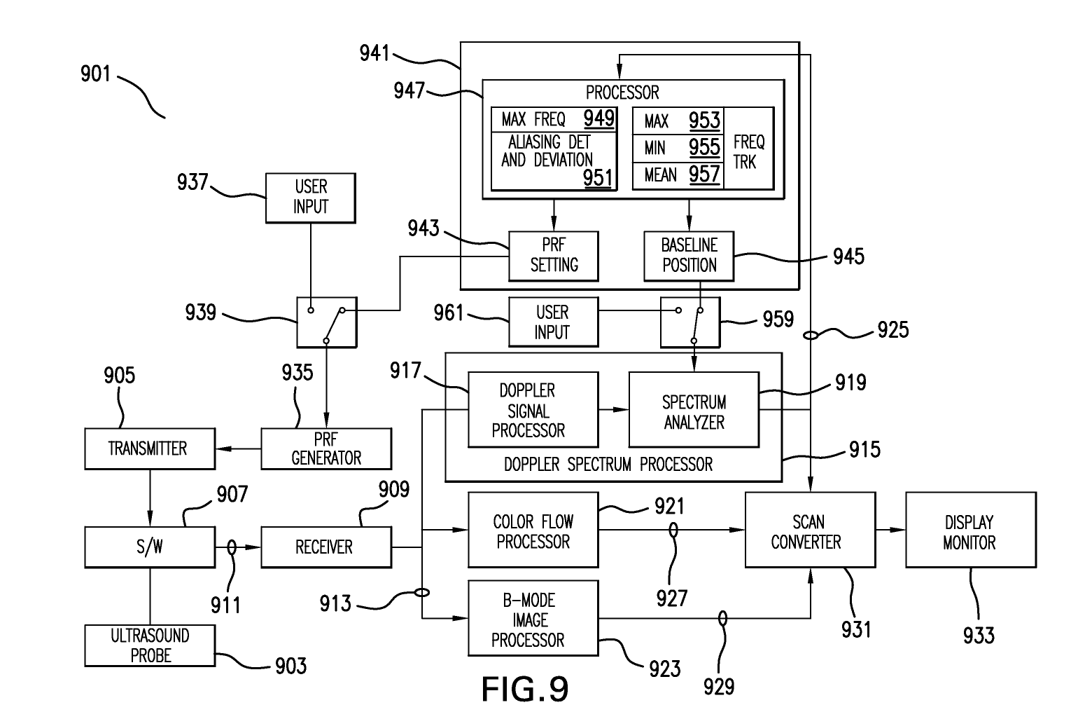

FIG. 9 shows an ultrasound system 901 with automatic baseline shifting and PRF setting. FIGS. 6, 7 and 8 show flow charts that describe various methods used by the system 901. An ultrasound signal is transmitted from an ultrasound probe 903 driven by a transmitter 905 through a transmit/receive switch 907. A receiver 909 receives the received ultrasound signal from the probe 903 through the switch 907 and processes the signal 911.

The processed signal 913 is coupled to a Doppler spectrum processor 915, a color flow processor 921, and a B-mode image processor 923. The Doppler spectrum processor 915 includes a Doppler signal processor 917 and a spectrum analyzer 919, and processes Doppler flow velocity signals and calculates and outputs a Doppler spectrum 925. The color flow processor 921 processes the received signal 913 and calculates and outputs velocity, power and variance signals 927. The B-mode image processor 923 processes the received signal 913 and calculates and outputs a B-mode image 929 or the amplitude of the signal by an amplitude detection.

The Doppler spectrum signals 925, color flow processor signals (velocity, power, and variance) 927 and B-mode processor signals 929 are coupled to a scan converter 931 that converts the signals to scan-converted signals. The scan converter 931 output is coupled to a display monitor 933 for displaying ultrasound images.

The processed signal 913 is coupled to a Doppler signal processor 917 for computing Doppler flow signals in the time domain. The Doppler flow signals are coupled to a spectrum analyzer 919 that converts the time domain Doppler signals into their spectrum frequency components 925. The frequency components, or spectrum 925, are indirectly coupled to a pulse repetition frequency (PRF) generator 935. The PRF generator 935 generates a pulse repetition frequency (PRF) depending on an input from either a manual user input 937 coupled to the PRF generator 935 through a switch 939 or from an automatic baseline shifting and PRF setting processor 941. The automatic baseline shifting and PRF setting processor 941 includes a PRF setting device 943, a baseline position device 945 and a processor 947 that may be implemented as a DSP, an FPGA, an ASIC or as discrete components. The processor 947 derives a baseline shift and/or a PRF setting that is coupled to the PRF generator 935. The baseline shift is either controlled by a user input 961 through a switch 959 or automatically by the baseline position device 945 through the switch 959. The switch 959 lets the user choose between a user input mode or an automatic mode.

The processor 947 includes engines that calculate a maximum frequency and a minimum frequency 949, detect aliasing and deviation 951, and track maximum 953, minimum 955 and mean 957 frequencies from the Doppler spectrum 925. The processor 947 optimizes imaging by analyzing the Doppler frequency spectrum 925 and generates PRF settings 943 and baseline zero frequency shifts 945 if necessary.

With reference to FIG. 6, in use, the ultrasound system 901 may use a default PRF for a specific application like cardiac, carotid, or liver imaging to observe the blood flow Doppler spectrum (step 602). A maximum PRF is the highest frequency range or the highest velocity range of the ultrasound system.

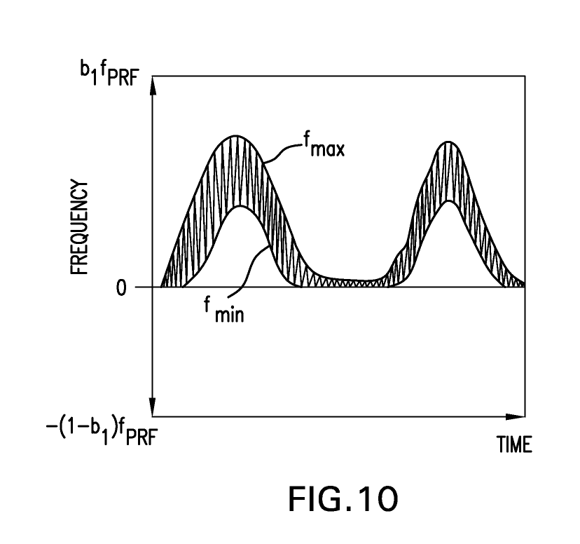

The Doppler spectrum image output 925 is typically a changing frequency spectrum over time as shown in FIG. 10, or a frequency (vertical axis) versus time (horizontal axis) with the power as the brightness. The brightness of the Doppler spectrum indicates the spectrum power at the frequency. Maximum Doppler frequencies are calculated 949 from the Doppler spectrum 925 and are tracked over time as a curve of maximum frequencies as shown in FIG. 10.

The maximum frequency engine 949 calculates a maximum frequency as a percentile frequency. The total area of the Doppler spectrum is first obtained by integration of powers in all frequencies, as shown in the denominator of the following expression,

$$\frac{{\int }_{0}^{{f}_{max}} p d f}{{\dot{\int }}^{ } p d f} = 0.999 \qquad ( 2 )$$

where p is the spectrum power (or a spectrum amplitude spectrum a, or a power raised to a power a^{b}, where b is a real number, or any signal derived from the amplitude). A percentile such as 99 or 99.9 percent is applied to the total area (i.e., the denominator of (2)) yielding a percentile area. The second integration (the numerator of (2)) begins at 0 frequency and ends when the integration reaches the percentile area. The maximum frequency is the frequency where the integration stops. In case of spectrum aliasing, (2) may not be satisfied even if the integration (numerator of (2)) reaches the maximum frequency range. In this case, the integration continues to the negative maximum frequency range and proceeds towards 0 frequency in the negative frequency range until (2) is satisfied.

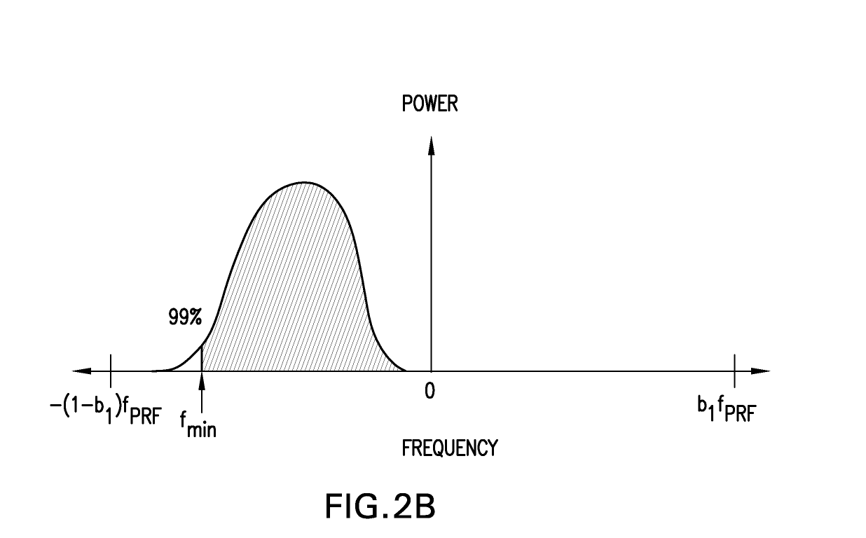

FIG. 2A shows a Doppler spectrum as frequency versus power plot at a given time. FIG. 2A shows a Doppler spectrum showing that the 99 percentile frequency represents the maximum frequency value fₘₐₓ for that spectrum sample (step 604) between positive and negative frequency range limits −(1−b₁)f_{PRF} to b₁f_{PRF}, where b₁ is a fraction between 0 and 1 and determines the position of the 0 frequency baseline and thus the positive and negative frequency ranges −(1−b₁)f_{PRF} to 0 and 0 to b₁f_{PRF}. If

$${b}_{1} = \frac{1}{2} ,$$

the positive and negative frequency ranges are equal. The maximum frequency values fₘₐₓ for each spectrum sample are tracked over time like that of a curve.

A noise reduction technique may be used to reduce noise from the Doppler spectrum 925. The Doppler spectrum power may be suppressed by a noise reduction gain control. The power spectrum may be replaced by an amplitude spectrum a, or a power raised to a power a^{b}, where b is a real number, or any signal derived from the amplitude.

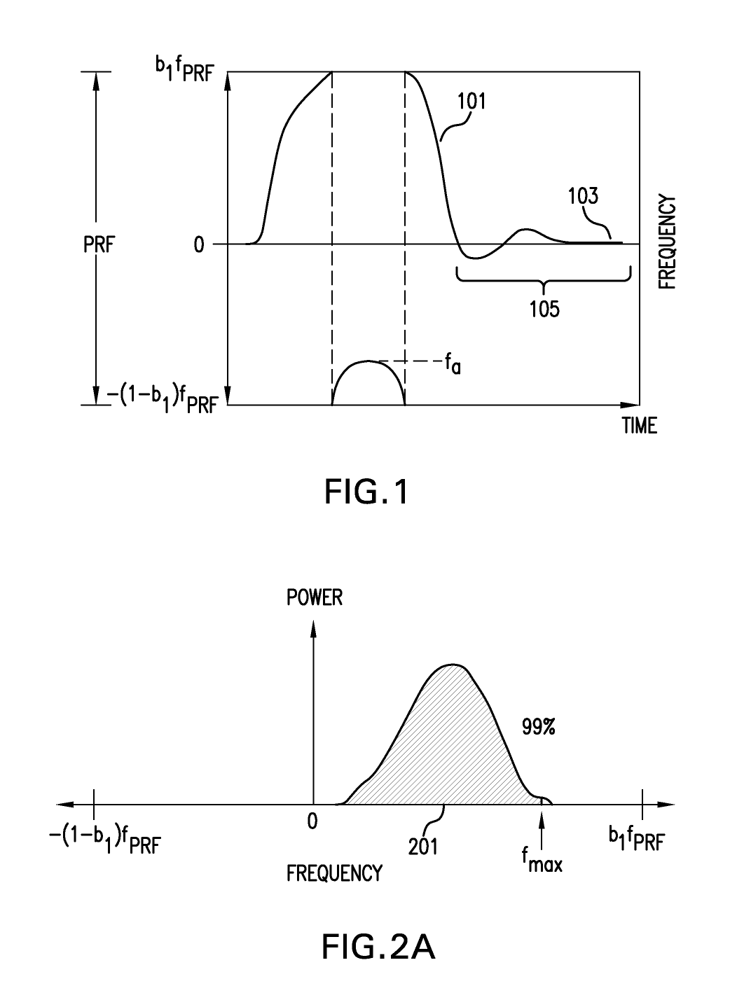

FIG. 1 shows a maximum frequency fₘₐₓ curve 101 that is aliased. The maximum frequency curve 101 may move in a positive or negative frequency direction with respect to a zero frequency baseline 103.

However, if a maximum frequency fₘₐₓ exceeds the PRF frequency range limits, the positive maximum frequency limit b₁f_{PRF} or the negative maximum frequency limit −(1−b₁)f_{PRF}, the frequencies greater than the frequency limits change (wrap) to the opposite maximum frequency regions as shown at b₁f_{PRF}. This sudden polarity change is detected by the aliasing detector and deviation engine 951 as aliasing (steps 606, 610). A change of polarity may occur in the absence of aliasing naturally near the baseline where the frequencies transition from positive to negative 105.

When aliasing is detected, a maximum frequency deviation fₐ corresponding to the magnitude of the wrapped frequency from either the maximum positive b₁f_{PRF} or negative −(1−b₁)f_{PRF}, frequency range limits is calculated by the deviation engine 951. In FIG. 1, the maximum deviation fₐ from the negative maximum frequency range −(1−b₁)f_{PRF} is calculated. When the PRF is too small and aliasing occurs, more than one frequency extreme may alias (frequency wrap) fₐ₁, fₐ₂, fₐ₃, . . . etc. The aliasing detector and deviation engine 951 detects each alias (frequency wrap) and compares all aliased frequencies to find the maximum frequency deviation fₐ during an observation period.

The maximum frequency deviation fₐ is used to offset the baseline 103 in either a positive or negative frequency direction depending on whether positive or negative frequencies are being aliased. A predetermined frequency safety margin fₛ may be added to the maximum frequency deviation fₐ to ensure that after a baseline 103 shift is implemented, no frequencies will be greater than the maximum positive b₁f_{PRF} or negative −(1−b₁)f_{PRF} frequency limits. A baseline shift is determined by

baseline shift=±(fₐ+fₛ). (3)

The sign in (3) indicates the direction of the baseline shift. Minus indicates a baseline shift in a negative frequency direction while plus indicates a baseline shift in a positive frequency direction.

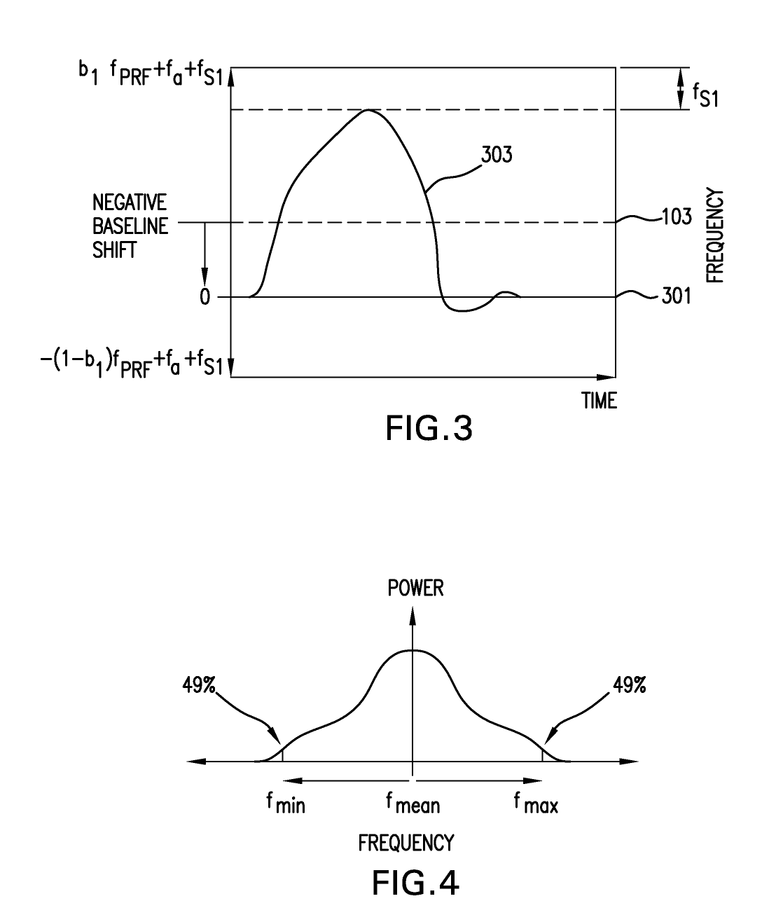

FIG. 3 shows the result of a baseline shift to the aliased maximum frequency fₘₐₓ curve 101 in FIG. 1. The baseline shift 301 adjusts the baseline in a positive or negative frequency direction to obtain a non-aliased maximum frequency fₘₐₓ curve 303. Since the maximum frequency deviation fₐ in FIG. 1 was detected in the negative frequency region, the direction from (3) is negative and the baseline 103 is displaced by the calculated baseline shift 303 that includes the maximum frequency deviation fₐ and predetermined frequency safety margin fₛ (3) (step 608). The method in FIG. 6 adjusts the baseline and maintains a constant PRF setting.

When a baseline is shifted, the positive and negative frequency ranges change with the baseline shift. After the baseline shift, the positive maximum frequency limit becomes b₁f_{prf}+fₐ+fₛ₁, while the negative maximum frequency limit becomes −(1−b₁)f_{prf}+fₐ+fₛ₁. For example, if the calculated baseline shift for FIG. 1 resulted in

$$- \frac{1}{4} {f}_{prf} , \qquad ( 3 )$$

the baseline 301 in FIG. 3 is shifted in a negative frequency direction by

$$\frac{1}{4} {f}_{prf} .$$

If the current PRF fraction b₁ was ½, meaning that the negative and positive frequency ranges are

$$- \frac{1}{2} {f}_{PRF} to 0 and 0 to \frac{1}{2} {f}_{PRF} ,$$

the new negative frequency range becomes

$$- \frac{1}{4} {f}_{PRF} to 0$$

and the new positive frequency range becomes

$$0 to \frac{3}{4} {f}_{PRF} .$$

Baseline shifting adjusts the PRF fraction b₁,

b_{1new}f_{PRF}=b_{1current}f_{prf}−baseline shift. (4)

Baseline shifting using the maximum frequency fₘₐₓ is described above to correct aliasing experienced at the positive frequency limit. This method applies at the negative frequency limit by using a minimum frequency fₘᵢₙ. A minimum frequency fₘᵢₙ is calculated as a percentile value. The total area of the Doppler spectrum is first obtained by integration of powers in all frequencies, as shown in the denominator of the following expression,

$$\frac{{\int }_{{f}_{min}}^{0} p d f}{\dot{\int } p d f} = 0.999 , \qquad ( 5 )$$

where p is the spectrum power (or a spectrum amplitude spectrum a, or a power raised to a power a^{b}, where b is a real number, or any signal derived from the amplitude). A percentile such as 99 or 99.9 percent is applied to the total area yielding a percentile area. The second integration (the numerator of (5)) begins at 0 frequency and ends when the integration reaches the percentile area as shown in FIG. 2B. The maximum frequency is the frequency where the integration stops. Baseline shifting using the maximum frequency is simply converted to the baseline shifting by the minimum frequency in case of aliasing involving the negative maximum frequency. Aliasing at the negative frequency range is detected when the minimum frequency changes (wraps) from the negative maximum frequency limit to the positive maximum frequency limit. The aliased portion will be corrected by the baseline shift in the opposite direction as previously described for aliasing at the positive maximum frequency range.

Furthermore, the maximum frequency and minimum frequency may be obtained in alternate methods as follows.

First, a mean frequency fₘₑₐₙ is obtained using

$${f}_{mean} = \frac{\int fp d f}{\int p d f} . \qquad ( 6 )$$

Then, maximum fₘₐₓ and minimum fₘᵢₙ frequencies are calculated as follows,

$$\begin{matrix}\frac{{\int }_{{f}_{mean}}^{{f}_{max}} p d f}{{\dot{\int }}_{ }^{ } p d f} = 0.499 , and & ( 7 ) \\ \frac{{\int }_{{f}_{min}}^{{f}_{mean}} p d f}{\dot{\int } p d f} = 0.499 . & ( 8 )\end{matrix}$$

where f is frequency and p is the Doppler spectrum power (or a spectrum amplitude spectrum a, or a power raised to a power a^{b}, where b is a real number, or any signal derived from the amplitude).

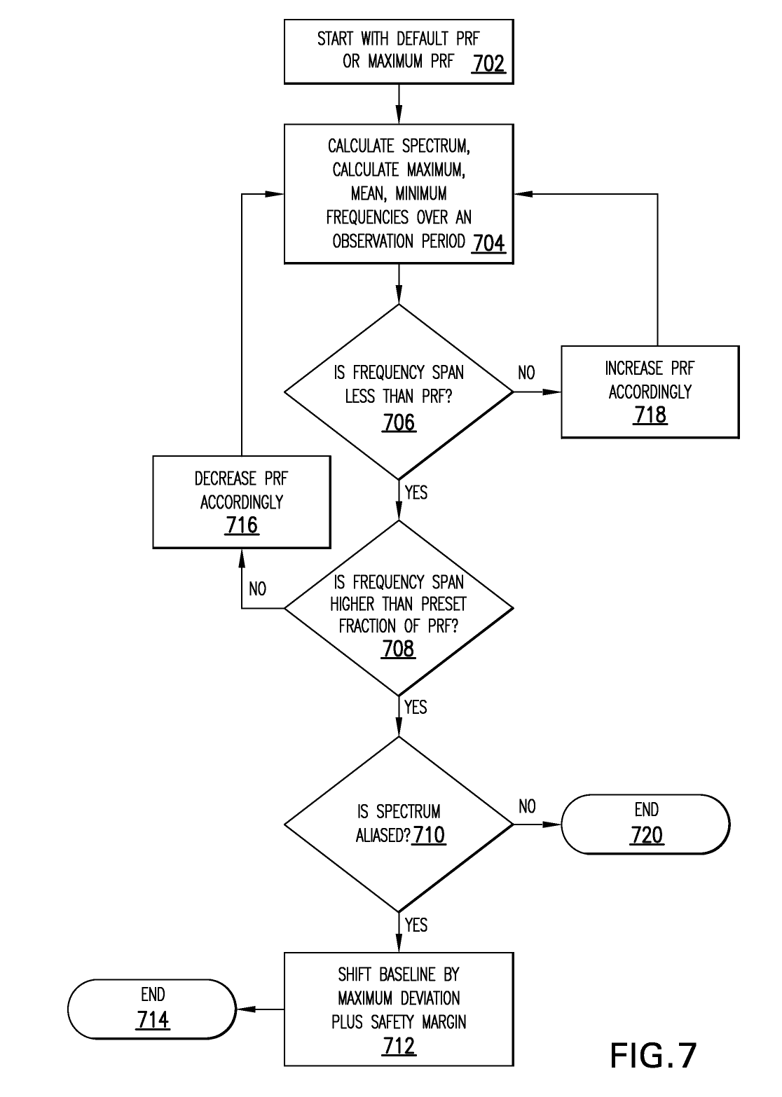

FIG. 7 shows a flow chart that describes a variant of baseline shifting that also includes adjusting the PRF setting. A maximum PRF may be used to first observe a blood flow Doppler spectrum without risking aliasing (step 702). Alternately, a preset PRF may be first used.

Similar to the above when calculating Doppler maximum frequencies fₘₐₓ, minimum Doppler frequencies fₘᵢₙ and mean Doppler frequencies fₘₑₐₙ are calculated by the maximum 953, minimum 955 and mean 957 engines. FIG. 4 shows a Doppler power spectrum identifying calculated maximum fₘₐₓ, minimum fₘᵢₙ, and mean fₘₑₐₙ frequency values for that spectrum. The maximum fₘₐₓ minimum fₘᵢₙ, and mean fₘₑₐₙ frequency values for each spectrum sample are tracked over time like curves.

The mean frequency fₘₑₐₙ may be calculated first as the first moment from a spectrum 925 as follows,

$${f}_{mean} = \frac{\int fp d f}{\int p d f} , \qquad ( 9 )$$

where f is frequency and p is the Doppler spectrum power (or a spectrum amplitude spectrum a, or a power raised to a power a^{b}, where b is a real number, or any signal derived from the amplitude).

After the mean frequency fₘₑₐₙ is calculated from the spectrum, maximum fₘₐₓ and minimum fₘᵢₙ Doppler frequencies are calculated.

The maximum fₘₐₓ and minimum fₘᵢₙ frequencies are calculated as percentile values of the spectrum from the calculated mean frequency fₘₑₐₙ value. For example, from the mean frequency fₘₑₐₙ, a maximum frequency fₘₐₓ of 49.9 percent may be calculated in the positive frequency direction starting from the mean frequency fₘₑₐₙ. The minimum frequency fₘᵢₙ is calculated similarly in the negative direction.

Together, the maximum fₘₐₓ and minimum fₘᵢₙ frequencies set a combined boundary of 99.8 percent of the total spectrum power as

$$\begin{matrix}\frac{{\int }_{{f}_{mean}}^{{f}_{max}} p d f}{\int p d f} = 0.499 , and & ( 10 ) \\ \frac{{\int }_{{f}_{min}}^{{f}_{mean}} p d f}{\int p d f} = 0.499 . & ( 11 )\end{matrix}$$

Since the mean frequency fₘₑₐₙ value is a weighted mean frequency of the spectrum, the maximum fₘₐₓ and minimum fₘᵢₙ frequency values are calculated by the maximum 953 and minimum 955 engines using (10) and (11), as long as the percentile values are less than 50 percent (step 704). Alternately, the maximum frequency and minimum frequency values may be calculated using (2) and (5), respectively.

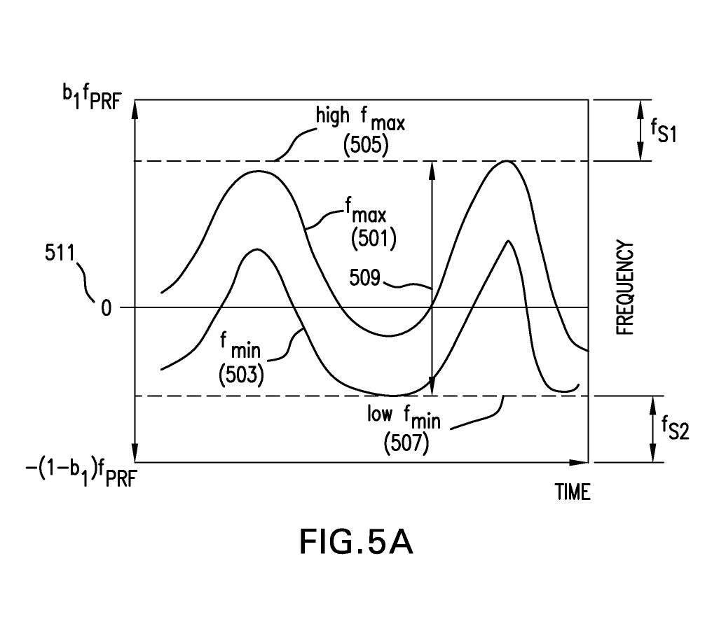

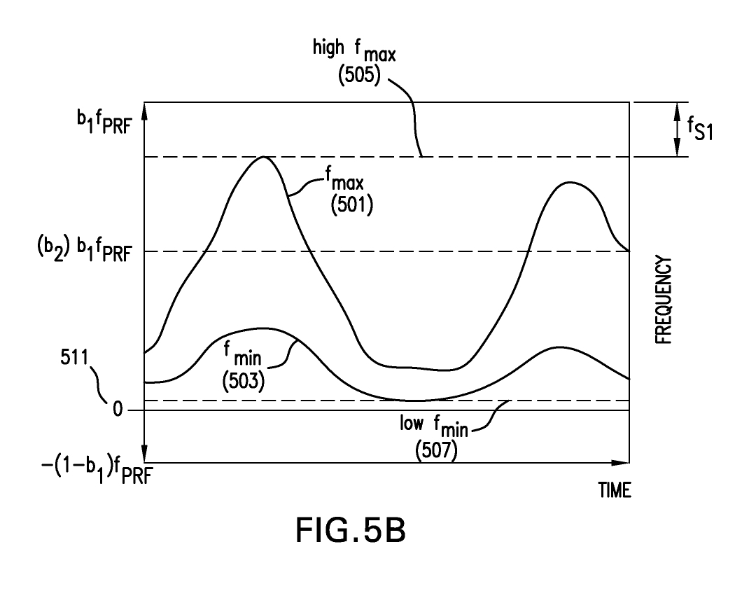

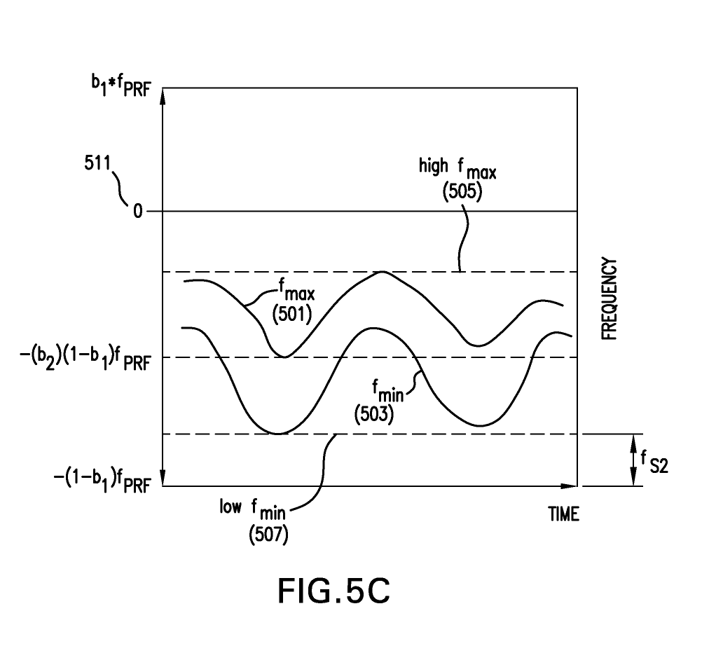

FIGS. 5A, 5B and 5C show calculated maximum fₘₐₓ 501 and minimum fₘᵢₙ 503 frequency values over time. These curves set high high fₘₐₓ 505 and low fₘᵢₙ 507 Doppler spectrum boundaries. The highest value high fₘₐₓ 505 of the maximum fₘₐₓ Doppler frequency curve and the lowest value low fₘᵢₙ of the minimum fₘᵢₙ Doppler frequency curve are captured and recorded.

If either the maximum fₘₐₓ or minimum fₘᵢₙ frequency curves experience aliasing (as in FIG. 1) during the observation period, the aliasing detector and deviation engine 951 continues tracking the maximum fₘₐₓ and minimum fₘᵢₙ frequency curves by adding the deviations experienced by each wrapped frequency to their respective clipped peaks. If clipping is detected at both the positive and negative maximum frequency ranges, the current PRF setting is too small.

The spectrum is unipolar positive if all frequency components residing in the positive frequency region which may include corrected aliased frequencies if the spectrum was once aliased. The spectrum is unipolar negative if all frequency components residing in the negative frequency region which may include corrected aliased frequencies if the spectrum was once aliased. The spectrum is bipolar if frequency components reside in both the positive and negative frequency regions after correcting aliasing if the spectrum was once aliased.

FIG. 5A shows a spectrum that is bipolar. A frequency span 509 between the highest maximum frequency high fₘₐₓ 505 and the lowest minimum frequency low fₘᵢₙ 507 is calculated and used to determine a new PRF for the best image display based on the observation period. The frequency span,

frequency span=(high fₘₐₓ)−(low fₘᵢₙ) (12)

may be considered the minimum PRF for the observed blood flow recording. Frequency safety margins fₛ₁ and fₛ₂ may be added to adjust the frequency span 509 ensuring adequate margins between the spectrum and the maximum frequency ranges.

adjusted frequency span=((high fₘₐₓ)−(low fₘᵢₙ))+fₛ₁+fₛ₂ (13)

The adjusted frequency span is compared with the current PRF setting (step 706). If the adjusted frequency span is greater than the current PRF setting 943,

adjusted frequency span>current PRF (14)

the current PRF setting 943 is increased by the processor 947 to a setting corresponding to the adjusted frequency span and output to the PRF generator 935 (step 718). If the adjusted frequency span is less than the current PRF setting, aliasing may not be occurring but the current PRF setting may be too large.

The adjusted frequency span is further compared with a fraction of the current PRF setting to reduce the PRF to a value that yields the best imaging display. If the PRF setting is too large for the blood velocity, the Doppler spectrum 925 display will be too small to accurately show the blood velocity.

A fraction of the current PRF is used as a low level threshold. A predetermined fraction between 0 and 1, for example ½, may be used as the fraction.

(fraction)(current PRF)<adjusted frequency span<current PRF (15)

If the adjusted frequency span is less than the fraction PRF, the Doppler spectrum image needs to be increased (step 708) in size. Therefore, the PRF 943 is decreased to the adjusted frequency span and output to the PRF generator 935 (step 716). The PRF setting is either decreased or increased until the adjusted frequency span is less than the current PRF setting, but greater than the fraction PRF.

FIG. 5B shows a spectrum that is unipolar positive. In this case, the highest maximum frequency high fₘₐₓ 501 plus a frequency safety margin fₛ₁ is used to determine a new PRF. The highest maximum frequency high fₘₐₓ 505 plus a frequency safety margin fₛ₁ is compared with the current positive maximum frequency limit b₁f_{PRF}. If the highest maximum frequency high fₘₐₓ 505 plus frequency safety margin fₛ₁ is greater than the current positive maximum frequency limit b₁f_{PRF} 943,

(high fₘₐₓ+fₛ₁)>b₁f_{PRF} (16)

the current PRF setting 943 is increased by the processor 947 to a setting corresponding to the highest maximum frequency high fₘₐₓ 501 plus frequency safety margin fₛ₁ and output to the PRF generator 935. If the highest maximum frequency high fₘₐₓ 501 plus a frequency safety margin fₛ₁ is less than the current positive maximum frequency limit b₁f_{PRF}, aliasing may not be occurring but the current PRF setting may be too large.

The highest maximum frequency high fₘₐₓ 501 plus frequency safety margin fₛ₁ is further compared with a fraction of the current positive maximum frequency limit b₁f_{PRF} to reduce the PRF to a value that yields the best imaging display. If the PRF setting is too small for the blood velocity to measure, aliasing will occur. However, if the PRF setting is too large for the blood velocity, the Doppler spectrum 925 display will be too small to accurately show the blood velocity.

A positive low level threshold b₂b₁f_{PRF}, where b₂ is a fraction between 0 and 1 is calculated and compared with the highest maximum frequency high fₘₐₓ 505 plus frequency safety margin fₛ₁.

b₂b₁f_{PRF}<(high fₘₐₓ+fₛ₁) (17)

If the highest maximum frequency high fₘₐₓ 505 plus frequency safety margin fₛ₁ is less than the current positive maximum frequency limit b₁f_{PRF}, the Doppler spectrum image needs to be increased in size. Therefore, the PRF 943 is decreased to the highest maximum frequency plus frequency safety margin high fₘₐₓ+fₛ₁ and output to the PRF generator 935. The PRF setting is either decreased or increased until the highest maximum frequency high fₘₐₓ 505 plus frequency safety margin fₛ₁ is less than the current positive maximum frequency limit b₁f_{PRF}, but greater than the positive low level threshold b₂b₁f_{PRF}.

FIG. 5C shows a spectrum that is unipolar negative. In this case, the lowest minimum frequency low fₘᵢₙ 507 plus a frequency safety margin fₛ₂ is used to determine a new PRF. The lowest minimum frequency low fₘᵢₙ 507 plus a frequency safety margin fₛ₂ is compared with the current negative minimum frequency limit −(1−b₁)f_{PRF}. If the absolute value of the lowest minimum frequency low fₘᵢₙ 507 plus frequency safety margin fₛ₂ is greater than the absolute value of the current negative maximum frequency limit −(1−b₁)f_{PRF} 943,

(|low fₘᵢₙ|+fₛ₂)>(1−b₁)f_{PRF} (18)

the current PRF setting 943 is increased by the processor 947 to a setting corresponding to the absolute value of the lowest minimum frequency low fₘᵢₙ 507 plus frequency safety margin fₛ₂ and output to the PRF generator 935. If the absolute value of the lowest minimum frequency low fₘᵢₙ 507 plus a frequency safety margin fₛ₂ is less than the absolute value of the current negative maximum frequency limit −(1−b₁)f_{PRF} aliasing may not be occurring but the current PRF setting may be too large.

The absolute value of the lowest minimum frequency low fₘᵢₙ 507 plus frequency safety margin fₛ₂ is further compared with a fraction of the absolute value of the current negative maximum frequency limit −(1−b₁)f_{PRF} to reduce the PRF to a value that yields the best imaging display. If the PRF setting is too small for the blood velocity to measure, aliasing will occur. However, if the PRF setting is too large for the blood velocity, the Doppler spectrum 925 display will be too small to accurately show the blood velocity.

A negative low level threshold −b₂(1−b₁)f_{PRF}, where b₂ is a fraction between 0 and 1 is calculated and compared with the lowest minimum frequency low fₘᵢₙ 507 plus frequency safety margin fₛ₂ which in turn is compared with the current negative maximum frequency limit −(1−b₁)f_{PRF}.

b₂(1−b₁)f_{PRF}<|low fₘᵢₙ|+fₛ (19)

If the absolute value of the lowest minimum frequency low fₘᵢₙ 507 plus a frequency safety margin fₛ₂ is less than the fraction of the absolute value of the current negative minimum frequency limit −(1−b₁)f_{PRF}, the Doppler spectrum image needs to be increased in size. Therefore, the PRF 943 is decreased to the absolute value of the lowest minimum frequency low fₘᵢₙ 507 plus a frequency safety margin fₛ₂ and output to the PRF generator 935. The PRF setting is either decreased or increased until the absolute value of the lowest minimum frequency low fₘᵢₙ 507 plus a frequency safety margin fₛ₂ is less than the absolute value of the current negative maximum frequency limit −(1−b₁)f_{PRF}, but greater than the absolute value of the negative low level threshold −b₂(1−b₁)f_{PRF}.

If aliasing is detected after adjusting the PRF regardless of whether the spectrum is bipolar, or positive or negative unipolar, (steps 710, 720, 712, 714), it may be corrected by baseline shifting as described above. Aliasing may occur after adjusting the PRF even if aliasing did not occur during the observation period when the PRF was being determined because the spectrum is not necessarily in the center of the frequency range. After decreasing the PRF, either high maximum or low minimum frequencies may exceed the corresponding limit.

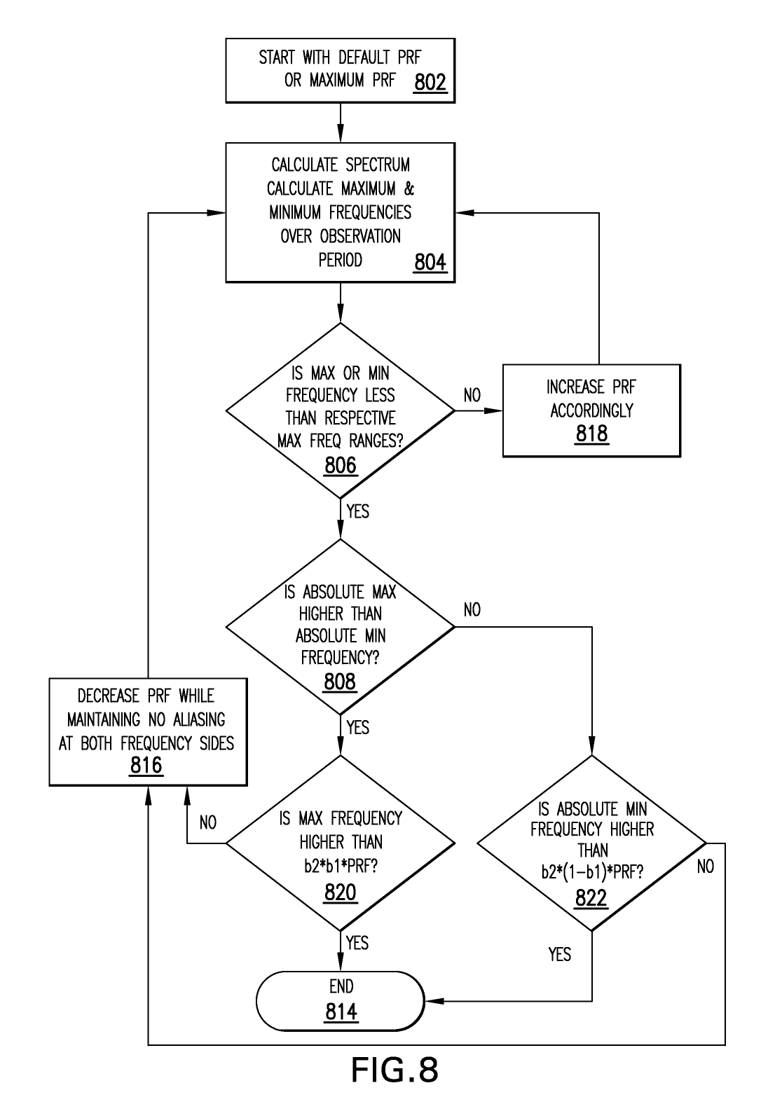

FIG. 8 shows a flow chart that describes a variant that adjusts the PRF setting but does not perform baseline shifting. The baseline may be fixed at a predetermined position anywhere between the positive maximum frequency range and the negative maximum frequency range. Initially, the PRF is set at either a default PRF value, or the maximum PRF (step 802). Ultrasound is transmitted at this PRF and the Doppler spectrum 925 processing is performed to obtain the Doppler spectrum.

The maximum fₘₐₓ and minimum fₘᵢₙ Doppler frequencies are calculated as described above in (10) and (11). The maximum fₘₐₓ and minimum fₘᵢₙ Doppler frequencies are monitored over an observation period (e.g. at least one cardiac cycle, heartbeat, or less than one cardiac cycle) and the highest value of the maximum fₘₐₓ Doppler frequency curve high fₘₐₓ and the lowest value of the minimum fₘᵢₙ Doppler frequency curve low fₘᵢₙ are recorded.

Frequency safety margins fₛ₁,fₛ₂ may be added to the absolute value of the highest maximum frequency high fₘₐₓ and the absolute value of the lowest minimum frequency low fₘᵢₙ,

|high fₘₐₓ|+fₛ₁, and (20)

|low fₘᵢₙ|+fₛ₂. (21)

(20) and (21) are used to find the best PRF setting.

The highest maximum frequency high fₘₐₓ plus frequency safety margin fₛ₁ is compared with the maximum positive frequency limit b₁f_{PRF}. If the highest maximum frequency high fₘₐₓ plus frequency safety margin fₛ₁ is greater than the positive maximum frequency limit b₁f_{PRF}, the PRF is increased to the level of the highest maximum frequency high fₘₐₓ plus frequency safety margin fₛ₁. Conversely, the absolute value of the lowest minimum frequency low fₘᵢₙ plus safety margin fₛ₂ is compared with the negative maximum frequency limit −(1−b₁)f_{PRF}. If the absolute value of the lowest minimum frequency low fₘᵢₙ plus frequency safety margin fₛ₂ is greater than the absolute value of the negative maximum frequency limit −(1−b₁)f_{PRF}, the PRF is increased to the absolute value of the lowest minimum frequency low fₘᵢₙ plus frequency safety margin fₛ₂ (steps 806, 818).

If the highest maximum frequency high fₘₐₓ plus frequency safety margin fₛ₁ is less than the positive maximum frequency limit b₁f_{PRF}, and the lowest minimum frequency low fₘᵢₙ plus safety margin fₛ₂ is less than the negative maximum frequency limit −(1−b₁)f_{PRF}, the absolute value of the highest maximum frequency high fₘₐₓ is compared with the absolute value of the lowest minimum frequency low fₘᵢₙ to determine which side of frequency component is dominant (step 808)

This comparison determines whether the positive or negative frequency region takes precedence. If

(|high fₘₐₓ|+fₛ₁)>(|low fₘᵢₙ|+fₛ₂) (22)

is true, the positive frequency region takes precedence and a positive low level threshold b₂b₁f_{PRF}, where b₂ is a fraction between 0 and 1 is calculated.

The highest maximum frequency high fₘₐₓ plus a frequency safety margin fₛ₁ is compared with the positive low level threshold b₂b₁f_{PRF} (step 820)

b₂b₁f_{PRF}<(high fₘₐₓ+fₛ₁). (23)

If (23) is satisfied, the PRF setting is complete (step 814). If the highest maximum frequency high fₘₐₓ plus frequency safety margin fₛ₁ (12) is less than the low level threshold b₂b₁f_{PRF}, the PRF is decreased to satisfy this condition while maintaining no aliasing at the negative frequency range (step 816). If aliasing starts to occur, the decreasing PRF stops even before satisfying this condition (23).

If (22) is not satisfied, the negative frequency region takes precedence and a negative low level threshold −b₂(1−b₁)f_{PRF} is calculated (step 808).

The absolute value of the lowest minimum frequency low fₘᵢₙ plus safety margin fₛ₂ is compared with the absolute value of the negative low level threshold −b₂(1−b₁)f_{PRF} (step 822)

(|low fₘᵢₙ|+fₛ₂)>b₂(1−b₁)f_{PRF}. (24)

If (24) is satisfied, the PRF setting is complete (step 814). If the absolute value of the lowest minimum frequency low fₘᵢₙ plus a frequency safety margin fₛ₂ is less than the absolute value of the low level threshold −b₂(1−b₁)f_{PRF}, the PRF is decreased to satisfy this condition (24) while maintaining no aliasing at the positive frequency side. If aliasing starts to occur, the decreasing PRF stops even before satisfying this condition (24).

One test determines whether the highest maximum frequency high fₘₐₓ plus a safety margin fₛ₁ is greater than the maximum positive frequency limit b₁f_{PRF} for aliasing, or, whether the absolute value of the lowest minimum frequency low fₘᵢₙ plus a safety margin fₛ₂ is greater than the absolute value of the minimum negative frequency limit −(1−b₁)f_{PRF}, for aliasing.

If the highest maximum frequency high fₘₐₓ plus a safety margin fₛ₁ is less than the maximum positive frequency limit b₁f_{PRF}, and, if the absolute value of the lowest minimum frequency low fₘᵢₙ plus a safety margin fₛ₂ is less than the absolute value of the minimum negative frequency limit −(1−b₁)f_{PRF}, another test is performed.

The other test determines if the highest maximum frequency high fₘₐₓ plus safety margin fₛ₁ is greater than the positive low level threshold b₂b₁f_{PRF} if the positive frequency is dominant (or (22) is true), or, whether the absolute value of the lowest minimum frequency low fₘᵢₙ plus safety margin fₛ₂ is greater than the absolute value of the negative low level threshold −b₂(1−b₁)f_{PRF} if the negative frequency is dominant (or (22) is false). This test ensures that the Doppler spectrum is large enough for the display. If the PRF is too high, the Doppler spectrum display suffers and is unacceptable for accurate clinical diagnosis. In this variant the baseline 103 is fixed and is not baseline shifted.

Since the baseline is not shifted, the decreasing PRF may cause aliasing in the spectrum in the frequency region that does not have precedence. For example, if positive frequencies have precedence, the above-described conditional tests adjust the current PRF based on positive frequency maximums and adjust the PRF accordingly. In decreasing the PRF, the negative portion associated with the spectrum may start to be aliased. When the negative portion of the spectrum starts aliasing, the decreasing PRF stops.

One or more embodiments of the present invention have been described. Nevertheless, it will be understood that various modifications may be made without departing from the spirit and scope of the invention. Accordingly, other embodiments are within the scope of the following claims.

What is claimed is:

1. A method for detecting and correcting aliasing in a Doppler frequency spectrum comprising:
receiving a Doppler frequency spectrum signal over time;
calculating maximum frequencies fₘₐₓ and minimum frequencies fₘᵢₙ from the Doppler frequency spectra
tracking the maximum fₘₐₓ and minimum fₘᵢₙ frequencies over time;
detecting whether aliasing is occurring from the maximum frequencies fₘₐₓ if frequencies in a positive frequency region change (wrap) to a negative frequency region, or detecting whether aliasing is occurring from the minimum frequencies fₘᵢₙ if negative frequencies in the negative frequency region change (wrap) to the positive region; and
if aliasing is detected, shifting a zero frequency baseline separating the negative and positive frequency regions of the Doppler spectrum in either a positive or negative direction according to a maximum frequency deviation fₐ.

2. The method according to claim 1 wherein the Doppler spectrum signal is from the group consisting of an amplitude spectrum a, a power spectrum a², or a power raised to a power a^{b}, where b is a real number.

3. The method according to claim 1 wherein the maximum frequencies fₘₐₓ are percentile values of the Doppler frequency spectra.

4. The method according to claim 1 wherein the minimum frequencies fₘᵢₙ are percentile values of the Doppler frequency spectra.

5. The method according to claim 1 further comprising determining the maximum frequency deviation fₐ from the magnitude of frequencies that have wrapped from one region (positive or negative) to the other (negative or positive).

6. The method according to claim 1 further comprising:
adding a frequency safety margin fₛ to the maximum frequency deviation fₐ; and
shifting the frequency spectrum baseline by the maximum frequency deviation plus safety margin ±(fₐ+fₛ).

7. A method of determining a pulse repetition frequency for an ultrasound system comprising:
receiving a Doppler frequency spectrum signal over time;
calculating maximum frequencies fₘₐₓ from the Doppler frequency spectra;
calculating minimum frequencies fₘᵢₙ from the Doppler frequency spectra;
tracking the maximum fₘₐₓ and minimum fₘᵢₙ frequencies over time;
capturing a highest value high fₘₐₓ of the maximum fₘₐₓ frequencies and a lowest value low fₘᵢₙ of the minimum fₘᵢₙ frequencies tracked;
comparing the highest value high fₘₐₓ and the lowest value low fₘᵢₙ to determine whether the maximum fₘₐₓ frequencies and minimum fₘᵢₙ frequencies are bipolar, or negative or positive unipolar;
if bipolar:
determining a frequency span based on a difference between the highest maximum frequency high fₘₐₓ and lowest minimum frequency low fₘᵢₙ;
comparing the frequency span to a current PRF setting value;
if the frequency span is greater than the current PRF setting value, increase the PRF setting value;
if the frequency span is less than a predetermined fraction of the current PRF setting value, decrease the PRF setting value; and
if the frequency span is less than the current PRF setting value but greater than the predetermined fraction of the current PRF, use the current PRF setting value;
if positive unipolar:
comparing the highest maximum frequency high fₘₐₓ with a current positive maximum frequency limit b₁f_{PRF}, wherein if the highest maximum frequency high fₘₐₓ is greater than the current positive maximum frequency limit b₁f_{PRF} the current PRF setting value is increased to a setting corresponding to the highest maximum frequency high fₘₐₓ;
if the highest maximum frequency high fₘₐₓ is less than a current positive maximum frequency limit b₁f_{PRF}, comparing the highest maximum frequency high fₘₐₓ with a low level threshold b₂b₁f_{PRF}, wherein if the highest maximum frequency high fₘₐₓ is less than the low level threshold b₂b₁f_{PRF}, the PRF is decreased until equal to the highest maximum frequency high fₘₐₓ; and
if negative unipolar:
comparing the absolute value of the lowest minimum frequency low fₘᵢₙ with the absolute value of a current negative maximum frequency limit −(1−b₁)f_{PRF}, wherein if the absolute value of the lowest minimum frequency low fₘᵢₙ is greater than the absolute value of the current negative maximum frequency limit −(1−b₁)f_{PRF}, the current PRF setting value is increased to a setting corresponding to the absolute value of the lowest minimum frequency low fₘᵢₙ;
if the absolute value of the lowest minimum frequency low fₘᵢₙ is less than the absolute value of the current negative maximum frequency limit −(1−b₁)f_{PRF}, comparing the absolute value of the lowest minimum frequency low fₘᵢₙ with the absolute value of a low level threshold −b₂(1−b₁)f_{PRF}, wherein if the absolute value of the lowest minimum frequency low fₘᵢₙ is less than the absolute value of the low level threshold −b₂(1−b₁)f_{PRF}, the PRF is decreased to equal the absolute value of the lowest minimum frequency low fₘᵢₙ.

8. The method according to claim 7 wherein the Doppler spectrum signal is from the group consisting of an amplitude spectrum a, a power spectrum a², or a power raised to a power a^{b}, where b is a real number.

9. The method according to claim 7 wherein the maximum frequencies fₘₐₓ are percentile frequencies of the Doppler frequency spectra.

10. The method according to claim 7 wherein the minimum frequencies fₘᵢₙ are percentile frequencies of the Doppler frequency spectra.

11. The method according to claim 7 further comprising:
detecting whether aliasing is occurring from the maximum frequencies fₘₐₓ if frequencies in a positive frequency region wrap to a negative frequency region;
detecting whether aliasing is occurring from the minimum frequencies fₘᵢₙ if negative frequencies in the negative frequency region wrap to the positive region; and
if aliasing is detected, shifting a zero frequency baseline separating the negative and positive frequency regions of the Doppler spectrum in either a positive or negative direction according to a maximum frequency deviation fₐ.

12. The method according to claim 7 further comprising:
detecting whether aliasing is occurring from the maximum frequencies fₘₐₓ if frequencies in a positive frequency region wrap to a negative frequency region;
detecting whether aliasing is occurring from the minimum frequencies fₘᵢₙ if frequencies in the negative frequency region wrap to the positive region; and
if aliasing is detected, adding aliased frequency deviations to the aliased maximum frequencies and to the aliased minimum frequencies.

13. The method according to claim 11 wherein the frequency deviation further comprises:
determining the maximum deviation fₐ from the magnitudes of frequencies that have wrapped from one region (positive or negative) to the other (negative or positive) to calculate the maximum frequencies fₘₐₓ and the minimum frequencies fₘᵢₙ.

14. The method according to claim 13 further comprising:
adding frequency safety margins to the maximum frequency deviation fₐ; and
shifting the frequency spectrum baseline by the maximum frequency deviation fₐ plus safety margins.

15. The method according to claim 7 further comprising:
adding frequency safety margins to the frequency span, highest maximum frequency high fₘₐₓ, or the absolute value of the lowest minimum frequency low fₘᵢₙ; and
comparing with the current PRF.

16. A method of determining a pulse repetition frequency for an ultrasound system comprising:
setting an initial pulse repetition frequency;
receiving a Doppler frequency spectrum signal over time;
calculating maximum frequencies fₘₐₓ from the Doppler frequency spectra;
calculating minimum frequencies fₘᵢₙ from the Doppler frequency spectra;
tracking the maximum fₘₐₓ and minimum fₘᵢₙ frequencies over time;
capturing a highest value high fₘₐₓ of the maximum frequencies fₘₐₓ and a lowest value low fₘᵢₙ of the minimum frequencies fₘᵢₙ tracked;
comparing the absolute value of the highest maximum value high fₘₐₓ with the absolute value of the lowest minimum frequency low fₘᵢₙ to determine whether the positive or negative frequency region takes precedence;
if the highest maximum value high fₘₐₓ is greater, the positive frequency region takes precedence and a positive low level threshold b₂b₁f_{PRF} is calculated; and
comparing the highest maximum frequency high fₘₐₓ with the positive maximum frequency limit b₁f_{PRF} and the positive low level threshold b₂b₁f_{PRF} wherein if the highest maximum frequency high fₘₐₓ is less than the positive low level threshold b₂b₁f_{PRF}, the PRF is decreased until the positive maximum frequency limit b₁f_{PRF} equals the highest maximum frequency high fₘₐₓ, or aliasing starts to occur at the negative maximum frequency limit −(1−b₁)f_{PRF} whichever comes first, and wherein if the highest maximum frequency high fₘₐₓ is greater than the positive maximum frequency limit b₁f_{PRF}, the PRF is increased to equal the highest maximum frequency high fₘₐₓ;
if the absolute value of the lowest minimum frequency low fₘᵢₙ is greater, the negative frequency region takes precedence and a low level threshold −b₂(1−b₁)f_{PRF} is calculated;
comparing the absolute value of the lowest minimum frequency low fₘᵢₙ with the absolute value of the negative maximum frequency limit −(1−b₁)f_{PRF} and the absolute value of the low level threshold −b₂(1−b₁)f_{PRF} wherein if the absolute value of the lowest minimum frequency low fₘᵢₙ is less than the absolute value of the low level threshold −b₂(1−b₁)f_{PRF}, the PRF is decreased until the absolute value of the negative maximum frequency limit −(1−b₁)f_{PRF} is the absolute value the lowest minimum frequency low fₘᵢₙ or aliasing starts to occur at the positive frequency limit whichever comes first, and wherein if the absolute value of the lowest minimum frequency low fₘᵢₙ is greater than the absolute value of the negative maximum frequency limit −(1−b₁)f_{PRF}, the PRF is increased to equal the lowest minimum frequency low fₘᵢₙ.

17. The method according to claim 16 further comprising adding frequency safety margins to the highest maximum frequency high fₘₐₓ and the absolute value of the lowest minimum frequency low fₘᵢₙ.

18. The method according to claim 16 wherein the positive maximum frequency limit b₁f_{PRF} and the negative maximum frequency limit −(1−b₁)f_{PRF} are determined by the PRF and a zero frequency baseline position which is fixed.

19. The method according to claim 16 wherein the maximum frequencies fₘₐₓ are percentile frequencies of the Doppler frequency spectra.

20. The method according to claim 16 wherein the minimum frequencies fₘᵢₙ are percentile frequencies of the Doppler frequency spectra.

21. The method according to claim 16 wherein the observation period may be less than a cardiac cycle or a long period of at least one cardiac period.

22. The method according to claim 16 wherein the maximum fₘₐₓ and minimum frequencies fₘᵢₙ are calculated from the Doppler spectra with or without a noise reduction.

23. A system for detecting and correcting aliasing in a Doppler frequency spectrum comprising:
means for receiving a Doppler frequency spectrum signal over time;
means for calculating maximum frequencies fₘₐₓ and minimum frequencies fₘᵢₙ from the Doppler frequency spectra;
means for tracking the maximum fₘₐₓ and minimum fₘᵢₙ frequencies over time;
means for detecting whether aliasing is occurring from the maximum frequencies fₘₐₓ if frequencies in a positive frequency region change (wrap) to a negative frequency region;
means for detecting whether aliasing is occurring from the minimum frequencies fₘᵢₙ if negative frequencies in the negative frequency region change (wrap) to the positive region; and
if aliasing is detected, means for shifting a zero frequency baseline separating the negative and positive frequency regions of the Doppler spectrum in either a positive or negative direction according to a maximum frequency deviation fₐ.

24. A system for determining a pulse repetition frequency for an ultrasound system comprising:
means for setting an initial pulse repetition frequency;
means for receiving a Doppler frequency spectrum signal over time;
means for calculating maximum frequencies fₘₐₓ from the Doppler frequency spectra;
means for calculating minimum frequencies fₘᵢₙ from the Doppler frequency spectra;
means for tracking the maximum fₘₐₓ and minimum fₘᵢₙ frequencies over time;
means for capturing a highest value high fₘₐₓ of the maximum frequencies fₘₐₓ and a lowest value low fₘᵢₙ of the minimum frequencies fₘᵢₙ tracked;
means for comparing the absolute value of the highest maximum value high fₘₐₓ with the absolute value of the lowest minimum frequency low fₘᵢₙ to determine whether the positive or negative frequency region takes precedence;
if the highest maximum value high fₘₐₓ is greater, the positive frequency region takes precedence and a positive low level threshold b₂b₁f_{PRF} is calculated; and
means for comparing the highest maximum frequency high fₘₐₓ with the positive maximum frequency limit b₁f_{PRF} and the positive low level threshold b₂b₁f_{PRF} wherein if the highest maximum frequency high fₘₐₓ is less than the positive low level threshold b₂b₁f_{PRF}, the PRF is decreased until the positive maximum frequency limit b₁f_{PRF} equals the highest maximum frequency high fₘₐₓ or aliasing starts to occur at the negative maximum frequency limit −(1−b₁)f_{PRF} whichever comes first, and wherein if the highest maximum frequency high fₘₐₓ is greater than the positive maximum frequency limit b₁f_{PRF}, the PRF is increased to equal the highest maximum frequency high fₘₐₓ;
if the absolute value of the lowest minimum frequency low fₘᵢₙ is greater, the negative frequency region takes precedence and a low level threshold −b₂(1−b₁)f_{PRF} is calculated;
means for comparing the absolute value of the lowest minimum frequency low fₘᵢₙ with the absolute value of the negative maximum frequency limit −(1−b₁)f_{PRF} and the absolute value of the low level threshold −b₂(1−b₁)f_{PRF} wherein if the absolute value of the lowest minimum frequency low fₘᵢₙ is less than the absolute value of the low level threshold −b₂(1−b₁)f_{PRF}, the PRF is decreased until the absolute value of the negative maximum frequency limit −(1−b₁)f_{PRF} is the absolute value the lowest minimum frequency low fₘᵢₙ or aliasing starts to occur at the positive frequency limit whichever comes first, and wherein if the absolute value of the lowest minimum frequency low fₘᵢₙ is greater than the absolute value of the negative maximum frequency limit −(1−b₁)f_{PRF}, the PRF is increased to equal the lowest minimum frequency low fₘᵢₙ.
